(19) United States
(12) Patent Application Publication
(10) Pub. No.: US 2003/0195606 A1
(43) Pub. Date: Oct. 16, 2003

(54) Bifurcation stent system and method

(75) Inventors: Charles J. Davidson, Winnetka, IL (US); Gil M. Vardi, Chesterfield, MO (US); Eric Williams, Evanston, IL (US); Stephen Kao, Mountain View, CA (US)

Correspondence Address: G. E. EHRLICH (1995) LTD., c/o ANTHONY CASTORINA, SUITE 207, 2001 JEFFERSON DAVIS HIGHWAY, ARLINGTON, VA, 22202, US

(73) Assignee: ADVANCED STENT TECHNOLOGIES, INC., A Delaware Corporation, Pleasanton, CA

(21) Appl. No.: 10/445,332

(22) Filed: May 27, 2003

Related U.S. Application Data
(63) Continuation of application No. 09/668,832, filed on Sep. 22, 2000, now abandoned.
(60) Provisional application No. 60/155,611, filed on Sep. 23, 1999.

(51) Int. Cl. A61F 2/06

(52) U.S. Cl. 623/1.11; 623/1.16

(57) ABSTRACT

Methods and apparatus for deploying a stent (20) in a bifurcated body lumen (10). The stent comprises a tubular body defining a lumen therethrough and having a side hole. The tubular body has a first portion (28) with a first wall mass and a second portion (26) with a second wall mass. The first wall mass is less than the second wall mass. When deployed, first portions of two stents overlap in a bifurcated body lumen. The side holes of the two stents are aligned with ostium of branch vessels (14, 16) at a bifurcated body lumen.

This application claims the benefit of U.S. Provisional Application No. 60/155,611 filed on Sep. 23, 1999, the complete disclosure of which is incorporated herein by reference.

### CROSS-REFERENCES TO RELATED APPLICATIONS

This application is being filed concurrently with related U.S. patent application Ser. No. ______ (Attorney Docket Number 019601-000420), entitled “Stent Range Transducers and Methods of Use”; and U.S. patent application Ser. No. ______ (Attorney Docket Number 019601-000410), entitled “Differentially Expanding Stent and Methods of Use”, the complete disclosures of which are incorporated herein by reference and filed at a date even herewith.

### TECHNICAL FIELD

The present invention relates to stents, stent systems, and methods for delivery and use thereof.

### BACKGROUND OF THE INVENTION

A type of endoprosthesis device, commonly referred to as a stent, may be placed or implanted within a vein, artery or other body lumen for treating occlusions, stenoses, or aneurysms of a vessel by reinforcing the wall of the vessel or by expanding the vessel. Stents have been used to treat dissections in blood vessel walls caused by balloon angioplasty of the coronary arteries as well as peripheral arteries and to improve angioplasty results by preventing elastic recoil and remodeling of the vessel wall. Two randomized multicenter trials have recently shown a lower restenosis rate in stent treated coronary arteries compared with balloon angioplasty alone (Serruys, P W et al., New England Journal of Medicine 331: 489-495 (1994) and Fischman, D L et al. New England Journal of Medicine 331:496-501 (1994)). Stents have been successfully implanted in the urinary tract, the bile duct, the esophagus and the tracheo-bronchial tree to reinforce those body organs, as well as implanted into the neurovascular, peripheral vascular, coronary, cardiac, and renal systems, among others. The term “stent” as used in this Application is a device which is intraluminally implanted within bodily vessels to reinforce collapsing, dissected, partially occluded, weakened, diseased or abnormally dilated or small segments of a vessel wall.

One of the drawbacks of conventional stents is that they are difficult to position in and around vessel bifurcations (branch points). Often treatment of diseased vessels at or near bifurcations requires placement of a stent in both a main vessel and a branch vessel at the bifurcation. In general, placement of stents in both the branch and main vessels involves positioning a main stent adjacent to a bifurcation such that an aperture in a side of the stent aligns with the ostium of a branch vessel. Then, a branch stent is positioned through the aperture and in the branch vessel. The branch stent is then attached to the main stent at the aperture.

Because this type of positioning and attachment can be difficult, it may provide suboptimal results. For example, if the branch stent is not properly attached, it may not adequately cover an area near the bifurcation. Further, if the branch stent remains substantially disposed within the main stent such that the main and branch stents overlap, there is risk that restenosis will occur due to metal burden.

In light of the foregoing, it would be desirable to provide advanced methods and/or apparatus to treat body lumens at or near bifurcations.

### SUMMARY OF THE INVENTION

The invention provides methods, systems and apparatus for positioning a stent in a bifurcated body lumen. The methods, systems and apparatus may be used to support three branches of a bifurcated body lumen. In one aspect of the invention, two identical stents can be used to support the three branches.

In one particular embodiment, a stent for placement in a bifurcated body lumen comprises a tubular body defining a lumen therethrough and having a side hole. The tubular body has a first portion with a first wall mass and a second portion with a second wall mass. The second wall mass is greater than the first wall mass.

In some embodiments of the invention the first wall mass is approximately one-half the second wall mass. Thus, when two first portions of two stents are overlapped, the wall mass of the combined two first portions approximately equals that of the second portion of a single stent.

In another embodiment of the invention, a stent system for placement in a bifurcated body lumen is provided. The bifurcated body lumen has a main vessel and first and second branch vessels. The system comprises a first stent body including a first portion, a second portion, and a side hole. The system further includes a second stent body with a first portion, a second portion, and a side hole. The first portion of the first stent body is disposed within and generally coaxially aligned with the first portion of the second stent body. The second portion of the first stent body extends through the side hole of the second stent body.

In yet another embodiment, a method for deploying a stent in a bifurcated body lumen is provided. The bifurcated body lumen includes a main vessel and first and second branch vessels. The method comprises providing first and second stent bodies each having a first portion, a second portion, and a side hole. The method further comprises positioning the first stent body within the bifurcated lumen such that the first portion is positioned within the main vessel and the second portion is positioned within the first branch vessel. The second stent body is positioned such that the first portion is generally aligned with and within the first portion of the first stent body within the main vessel, and the second portion extends through the side hole of the first stent body and into the second branch vessel. Both the first and second stent bodies are expanded.

Some embodiments of the method involve aligning the side hole of the first stent body with an ostium of the second branch vessel. Also, alignment of the side hole of the second stent body with an ostium of the first branch vessel is provided.

In still another embodiment, a kit comprising a stent along with instructions for use is provided. The instructions set forth a method for positioning the stent in a bifurcated body lumen.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings.

### BRIEF DESCRIPTION OF THE DRAWINGS

FIG. 1 illustrates a Y-shaped bifurcation in a body lumen for treatment with apparatus, systems and methods of the present invention;

FIGS. 2A and 2B depict overall views of two embodiments of a stent according to the present invention;

FIGS. 3A through 3C illustrate three embodiments each providing a differential wall mass between portions of the stent illustrated in FIGS. 2A and 2B;

FIGS. 4A through 4C show a “rolled out” view of three alternative strut patterns which can be used according to the present invention;

Figs. 5A through 5C illustrate an embodiment comprising stent placement in the Y-shaped bifurcation of FIG. 1; and

FIG. 6 shows a kit including a stent according to the present invention.

### DESCRIPTION OF THE SPECIFIC EMBODIMENTS

The invention provides methods, systems and apparatus for positioning a stent in a bifurcated body lumen. The methods, systems and apparatus may be used to support three or more branches of a bifurcated body lumen.

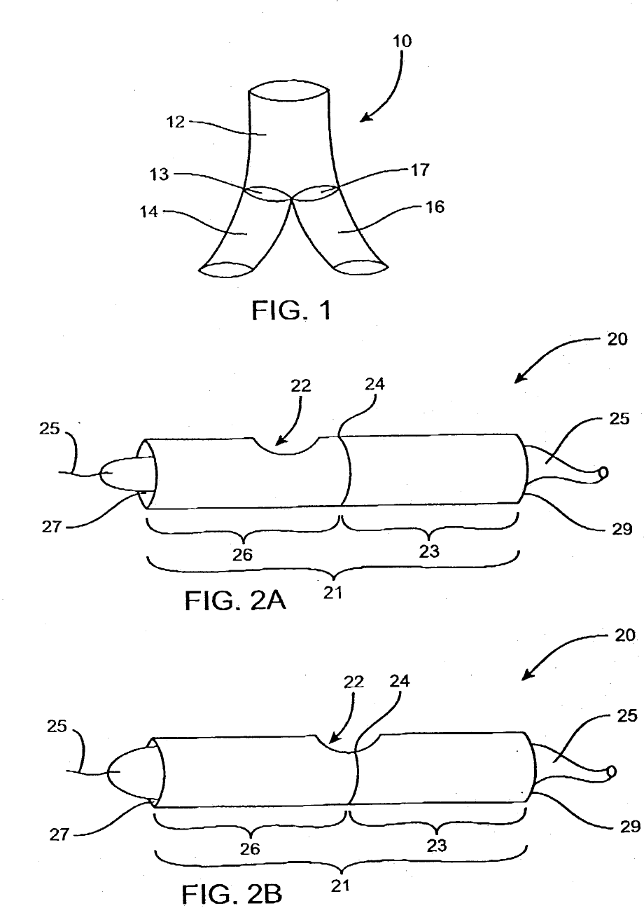

Applications of the invention include insertion into a body lumen including, among others, the cardiac, coronary, carotid artery, renal, peripheral vascular, gastrointestinal, pulmonary, urinary and neurovascular systems and the brain. The invention is particularly useful in applications involving Y-shaped bifurcations as illustrated in FIG. 1. A Y-shaped bifurcation 10 includes a main vessel 12, a left vessel 14 defining a left ostium 15, and a right vessel 16 defining a right ostium 17. It will be appreciated by those skilled in the art that left and right are arbitrary terms, and other configurations are within the scope of the present invention.

Referring now to FIG. 2A, an embodiment of a stent 20 includes an outer wall 21, a distal orifice 27, and a proximal orifice 29. Outer wall 21 includes a distal portion or end 26 and a proximal portion or end 28. Further, an interface 24 exists at a junction of distal end 26 and proximal end 28. Use of the term wall is not intended to limit stent 20 to solid, non-porous walls. Stent outer wall 21 preferably comprises a mesh-like structure as further described below. In this embodiment, stent 20 includes a side hole 22 formed in distal end 26. In an alternative embodiment shown in FIG. 2B, stent 20 includes side hole 22 formed partially in distal end 26 and partially in proximal end 28.

In some embodiments, a balloon 25 is disposed through the center of outer wall 21. As balloon 25 is inflated, it exerts pressure on outer wall 21 causing stent 20 to expand. It should be appreciated that other devices for exerting pressure on outer wall 21 can be used. Alternatively, in one embodiment, no device for exerting pressure on outer wall 21 is required as stent 20 is designed to expand without application of pressure, such as when stent 20 is released from a sheath.

In one embodiment of the invention, two identical stents 20 are positioned in Y-shaped bifurcation 10 and subsequently deployed. The stents 20 are positioned such that each of main vessel 12, left vessel 14, and right vessel 16 are supported near the bifurcation. When positioned, the two stents 20 overlap at their proximal ends 28 in main vessel 12. Distal end 26 of one stent is disposed in left vessel 14 and distal end 26 of the other stent is disposed in right vessel 16.

Distal end 26 of stent 20 is comprised of sufficient wall mass to support either left vessel 14 or right vessel 16 when deployed in the respective vessel. In contrast, wall mass of proximal end 28 is less than distal end 26. The reduced wall mass is designed such that when two proximal ends 28 overlap in main vessel 12, their combined wall mass is sufficient to support main vessel 12. Further, the wall mass of proximal end 28 is designed such that overlapping two proximal ends 28 in main vessel 12 does not cause metal burden on the stented body. In one particular embodiment, the wall mass of distal end 26 is approximately twice the wall mass of proximal end 28. Thus, when two proximal ends 28 overlap in main vessel 12, the wall mass in main vessel 12 approximately equals the wall mass in either left vessel 14 or right vessel 16.

Thus, the invention advantageously allows for overlapping stents near a bifurcation without causing metal burden on the stented body. This elimination of metal burden reduces the risk of restenosis.

As used herein, wall mass indicates a material density per surface area of outer wall 21. Accordingly, wall mass is a function of the material and/or geometry used to form outer wall 21. For example, increasing the thickness of outer wall 21 results in an increased wall mass. Further, using a higher density material also increases wall mass. Stent 20 may comprise, but is not limited to, stainless steel, nitinol, titanium, and the like.

As used herein, side hole 22 in stent 20 is a relatively large hole which is intended to be aligned with the ostium of a branch vessel. Such a side hole is separate from any of the multiple passageways extending through the side of stent 20 between struts in the stent geometry. Accordingly, side hole 22 is a hole which is understood to be larger than other passages through stent 20. In some embodiments, side hole 22 is defined by a band of continuous material which defines the perimeter of side hole 22. This continuous band of material preferably comprises discontinuities over its length so that the area of side hole 22 expands together with the expansion of stent 20.

It should be appreciated that the location of side hole 22 relative to distal end 26 and proximal end 28 can be varied. In some embodiments, side hole 22 is located such that only areas of outer wall 21 with reduced wall mass will overlap corresponding areas of another stent when side hole 22 is aligned with the ostium of a branch vessel.

It also should be appreciated that two identical stents 20 can be used to support main vessel 12, left vessel 14, and right vessel 16 near a bifurcation. Using two identical stents 20 reduces both manufacturing costs and insertion complexity. Further, two identical delivery systems may be used which further reduces manufacturing costs and insertion complexity.

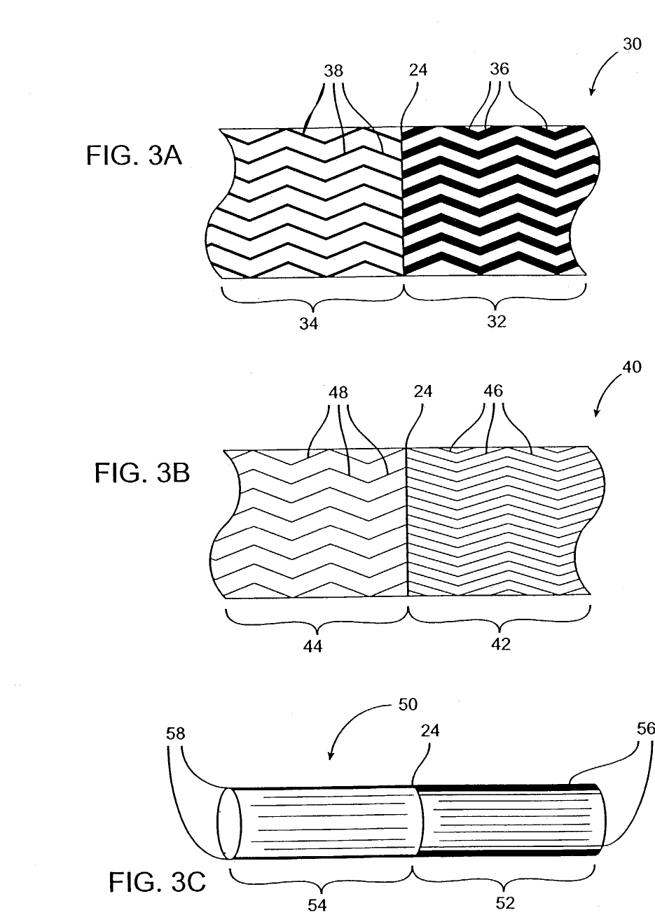

Three embodiments for providing a differential wall mass between proximal end 28 and distal end 26 are illustrated in FIGS. 3A through 3C. It should be recognized that these forms are merely illustrative and that many other embodiments for providing differential wall mass are possible according to the present invention.

FIG. 3A illustrates an embodiment of a portion 30 of stent 20. Portion 30 includes interface 24 at a junction between a distal portion 32 and a proximal portion 34. While junction 24 is depicted as a linear junction 24, junction 24 may have other shapes, including irregular and nonlinear shapes in this and other embodiments. Distal portion 32 is formed from struts 36 and proximal portion 34 is formed from struts 38. Struts 36 are similar in thickness, but wider than struts 38. Due to the larger width of struts 36, the wall mass of distal portion 32 is greater than the wall mass of proximal portion 34. In a preferred embodiment, struts 36 are of similar thickness and approximately twice as wide as struts 38. Thus, when two proximal portions 34 overlap, their combined wall mass is roughly equivalent to the wall mass at distal portion 32.

FIG. 3B illustrates an embodiment of a portion 40 of stent 20. Portion 40 includes interface 24 at a junction between a distal portion 42 and a proximal portion 44. Distal portion 42 is formed from struts 46 and proximal portion 44 is formed from struts 48. The geometry of struts 46 and 48 are similar, but the density per surface area of struts 46 is higher than a corresponding density for struts 48. This density of struts per surface area is also known as cell density. Due to the higher cell density in distal portion 42, distal portion 42 has a higher wall mass than proximal portion 44. In a preferred embodiment, the cell density in distal portion 42 is approximately twice the cell density in proximal portion 44. Thus, when two proximal portions 44 overlap, their combined wall mass is roughly equivalent to the wall mass at distal portion 42.

FIG. 3C illustrates an embodiment of a portion 50 of stent 20. Portion 50 includes interface 24 at a junction between a distal portion 52 and a proximal portion 54. Distal portion 52 is formed from struts 56 and proximal portion 54 is formed from struts 58. For clarity of illustration, only struts 56 and 58 on the top and bottom of stent 20 are shown, however, it should be understood that proximal portion 54 includes other struts 58 and that distal portion 52 includes other struts 56. Struts 56 are similar in width, but wider than struts 58. Thus, outer wall 21 of stent 20 is thicker at distal portion 52 than at proximal portion 54. Due to the greater thickness, the wall mass of distal portion 52 is greater than the wall mass of proximal portion 54. In a preferred embodiment, struts 56 are of similar width and approximately twice as thick as struts 58. Thus, when two proximal portions 54 overlap, their combined wall mass is roughly equivalent to the wall mass at distal portion 52.

From the foregoing discussion, it should be apparent that many combinations of materials, strut structure, and strut geometry can be used in accordance with the invention. For example, a structure including struts at proximal end 28 that are both wider and thicker than struts at distal end 26 could be used to provide a differential wall mass.

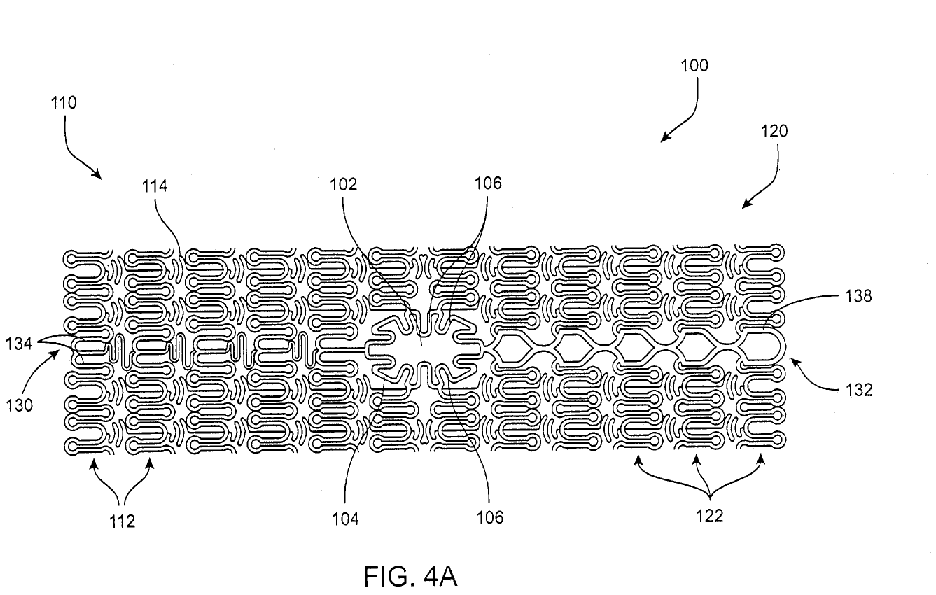

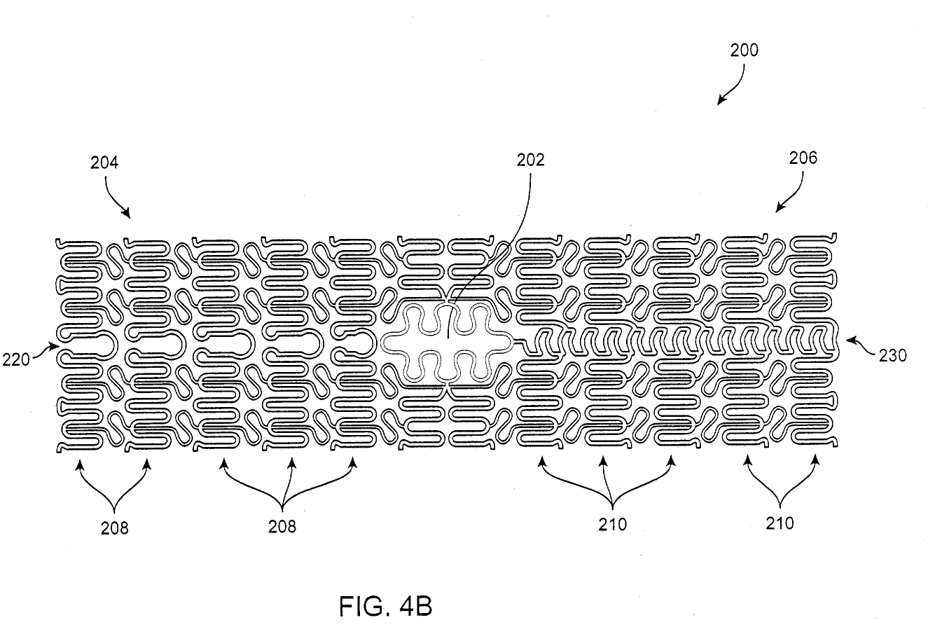

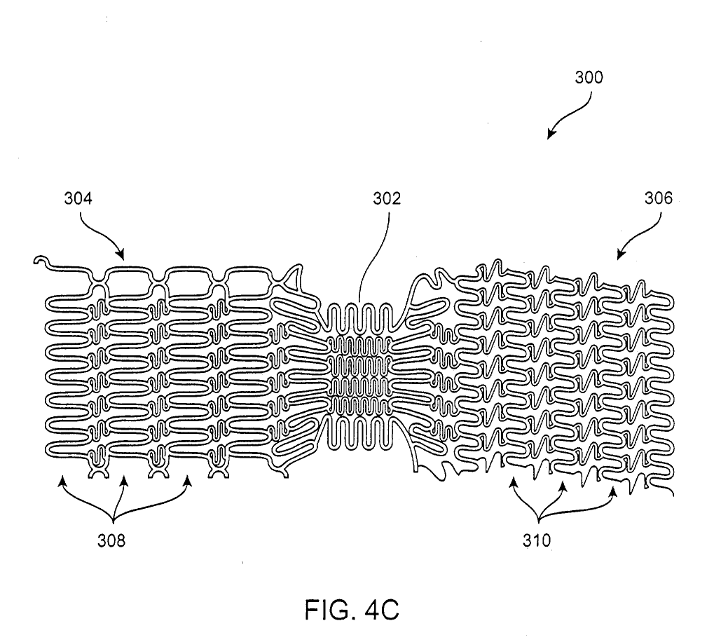

Further, it should be recognized that a number of strut patterns can be used to form both distal end 26 and proximal end 28. For example, FIGS. 4A through 4C illustrate three alternative strut patterns which can be used according to the invention. FIGS. 4A through 4C and the corresponding written description are adapted from U.S. patent application Ser. No. 09/600,348 (Attorney Docket No. 19601-000120), the complete disclosure of which is incorporated herein by reference.

Referring to FIG. 4A, a stent pattern 100 is illustrated in a “rolled out” view, i.e., a tubular stent is broken along an axial line and then rolled out to show stent pattern 100. Stent pattern 100 is illustrated prior to expansion. Stent pattern 100 includes a side hole 102 defined by a continuous band 104 having a plurality of loops 106 projecting into the open interior of side hole 102. Loops 106 are an integral part of band 104 and will, prior to expansion or opening, lie within the cylindrical envelope of the tubular body of stent 20. A distal portion 110 of stent pattern 100 lies on one side of side hole 102 and is defined by a plurality of serpentine rings 112. Serpentine rings 112 are joined by axial spring structures 114 so that stent pattern 100 may be bent as stent 20 is positioned and/or deployed. A proximal portion 120 of stent pattern 100 is formed on the other side of side hole 102. Proximal portion 120 is defined by a plurality of serpentine rings 122 which are generally similar in structure to rings 112 of distal portion 110. Each of the portions 110 and 120 include an axial spine 130 and 132, respectively. Axial spine 130 of distal portion 110 comprises simple W-shaped structures including outermost struts 134 which open at relatively low expansion force on the adjoining hinge regions. In contrast, axial spine 132 of proximal portion 120 comprises box elements 138 which require greater expansion force to open. Thus, during deployment, distal portion 110 will yield first to allow partial opening before proximal portion 120 begins to open.

According to the invention, stent pattern 100 can be formed such that rings 112 are either thicker, wider, or formed at a higher cell density than rings 122. Alternatively, any combination of thickness, width or cell density can be used to provide a differential wall mass between proximal portion 120 and distal portion 110.

A second stent pattern 200 is illustrated in FIG. 4B. A side hole 202 is formed from a continuous band of material, generally as described in relation to FIG. 4A. A distal portion 204 and a proximal portion 206 of stent pattern 200 each comprise a plurality of serpentine ring structures 208 and 210, respectively. While the specific geometries differ, the structures of stent patterns 100 and 200 are generally the same, except for distal spine portion 220 and proximal spine portion 230. Distal spine portion 220 comprises a simple U-shaped loop having a pair of struts joined by a simple C-shaped hinge region. Distal spine portion 220 will thus open at relatively low expansion forces. In contrast, proximal spine portion 230 comprises a serpentine element which allows for axial expansion but does not permit radial expansion. Thus, distal portion 204 will begin opening at much lower expansion forces or pressures than will proximal portion 206.

As with stent pattern 100 and according to the invention, stent pattern 200 can be formed such that rings 208 are either thicker, wider, or formed at a higher cell density than rings 210. Alternatively, any combination of thickness, width or cell density can be used to provide a differential wall mass between proximal portion 206 and distal portion 204.

A third stent pattern 300 is illustrated in FIG. 4C. Stent pattern 300 comprises a side hole 302 (which is shown in halves in the illustration), a distal portion 304, and a proximal portion 306. Distal portion 304 and proximal portion 306 each comprise serpentine rings 308 and 310, respectively. Serpentine rings 308 and 310 have different characteristics. More specifically, serpentine rings 308 have axially aligned struts joined by simple hinge regions. The length of the struts is relatively long (compared to those in the proximal region 306 as described below) so that the rings will open at a lower expansion pressure or force. In contrast, serpentine rings 310 of proximal portion 306 have relatively short axial struts defined by hinge regions each having two bands. Such structures require greater expansion force than do serpentine rings 308 of the distal portion 304. Similar to stent patterns 100 and 200, stent pattern 300 can be formed such that wall mass is greater in distal portion 304 than proximal portion 306.

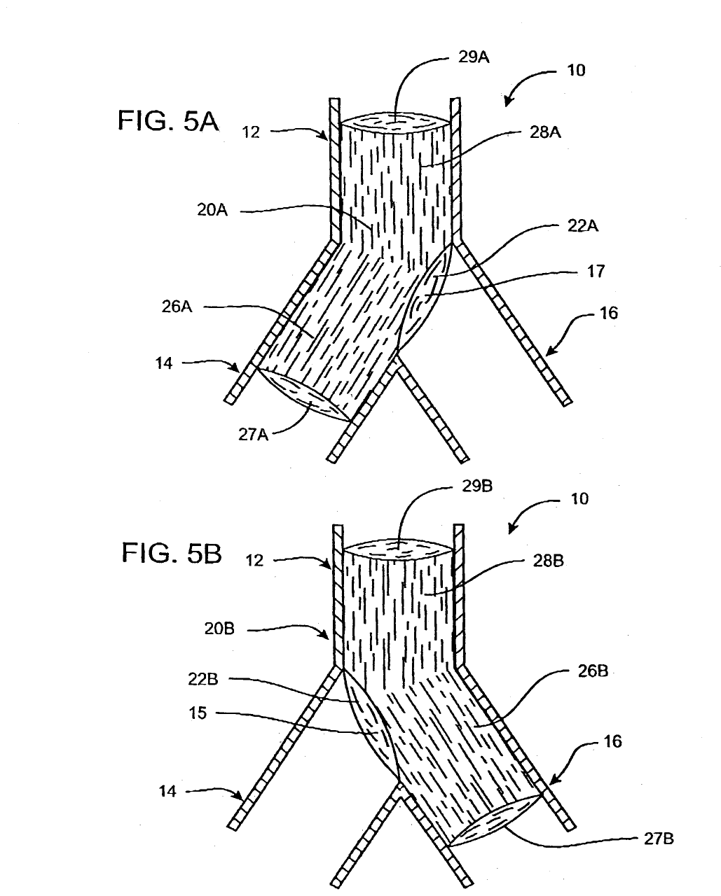

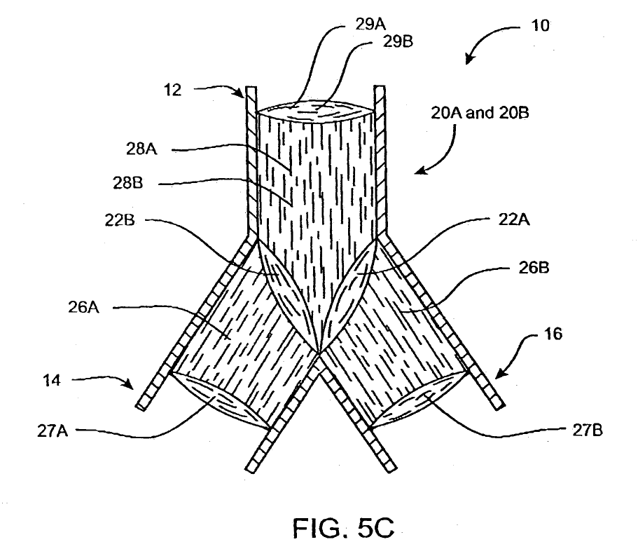

Referring now to FIGS. 5A through 5C, a method of deploying stents 20 in Y-shaped bifurcation 10 according to the present invention is described. Two stents 20A and 20B are used to support main vessel 12, left vessel 14, and right vessel 16.

Referring to FIG. 5A, stent 20A is positioned such that proximal end 28A is disposed in main vessel 12 and distal end 26A is disposed in left vessel 14. Side hole 22A is aligned with right ostium 17. Once this alignment is achieved, stent 20A is deployed by expanding the outer wall of stent 20A until both proximal end 28A and distal end 26A contact main vessel 12 and left vessel 14, respectively. After deployment, a passage exists through stent 20A connecting main vessel 12, left vessel 14, and right vessel 16. The passage connecting main vessel 12 and left vessel 14 includes distal orifice 27A and proximal orifice 29A. The passage connecting main vessel 12 and right vessel 16 includes proximal orifice 29A and side hole 22A.

As should be recognized, stent 20A can include structure which conforms to the geometry of main vessel 12 and left vessel 14. The structure further provides access to right vessel 16 through side hole 22A. Thus, when stent 20A is expanded radially outward, it conforms to main vessel 12 and left vessel 14. Such conformity allows stents according to the present invention to fit into different sizes of main 12, left 14, and right 16 vessels.

After deployment of stent 20A, stent 20B is positioned in main vessel 12 and right vessel 16. FIG. 5B illustrates positioning of stent 20B. For clarity of illustration, stent 20A which is positioned according to FIG. 5A is not shown. Stent 20B is positioned such that distal end 26B extends through side hole 22A (not shown) of stent 20A and into right vessel 16. Proximal end 28B is positioned within proximal end 28A (not shown) of stent 20A and main vessel 12. Side hole 22B is aligned with left ostium 15 of left vessel 14. Once this alignment is achieved, stent 20B is deployed by expanding the outer wall of stent 20B until distal end 26B contacts right vessel 16 and proximal end 28B contacts proximal end 28A (not shown) of stent 20A. Thus, a passage exists through stent 20B connecting main vessel 12, left vessel 14 and right vessel 16.

FIG. 5C shows both stent 20A and stent 20B deployed in Y-shaped bifurcation 10. Using the prior discussion in conjunction with FIG. 5C, a passage through stents 20A and 20B connecting main vessel 12, left vessel 14, and right vessel 16 is shown. The passage connecting main vessel 12 and left vessel 14 includes distal orifice 27A, side hole 22B, proximal orifice 29A, and proximal orifice 29B. The passage connecting main vessel 12 and right vessel 16 includes distal orifice 27B, side hole 22A, proximal orifice 29A, and proximal orifice 29B,. Further, it is shown that proximal portions 28A and 28B overlap in main vessel 12, while distal portions 26A and 26B are disposed in left vessel 14 and right vessel 16, respectively. As proximal portions 28A and 28B comprise reduced wall mass, overlapping portions 28A and 28B provides similar coverage to main vessel 12 as is provided in left vessel 14 and right vessel 16. Of course, it should be appreciated that stent 20B can be positioned and deployed prior to positioning and deploying stent 20A. Further, it should be appreciated that either stent 20A and/or stent 20B can be positioned by advancing through main vessel 12, left vessel 14, or right vessel 16 toward Y-shaped bifurcation 10.

Another embodiment provides for positioning stent 20A in main vessel 12 and left vessel 14 followed by partial deployment of stent 20A. After stent 20A is partially deployed, stent 20B is positioned such that proximal end 28B is disposed within proximal end 28A of stent 20A and distal end 26B is disposed in right vessel 16. After positioning stent 20B, both stent 20A and stent 20B are fully deployed.

In yet another embodiment, stent 20A includes a balloon 25 disposed therethrough. Stent 20A, including balloon 25 are positioned according to the discussion above. Balloon 25 is then inflated causing stent 20A to deploy. Balloon 25 is then deflated and removed from stent 20A. Stent 20B, including a similar balloon 25 or the same balloon 25, is then positioned according to the previous discussion. Balloon 25 is then inflated causing stent 20B to deploy. After deployment, balloon 25 is removed and stents 20A and 20B remain deployed as shown in FIG. 5C.

In still another embodiment, stent 20B is partially disposed within stent 20A prior to positioning within vessel 12. Together, both stent 20A and 20B are positioned in main vessel 12 near Y-shaped bifurcation 10. Stent 20A, with stent 20B partially disposed within, is located such that side hole 22A aligns with right ostium 17. Stent 20A is then partially expanded. Then, stent 20B is positioned through side hole 22A such that proximal end 28B is approximately concentric with and within proximal end 28A and distal end 26B is located in right vessel 16. Both stent 20A and 20 B are then fully expanded.

In another embodiment (not shown in the figures), a first stent 20 including a first and a second side hole could be positioned in main vessel 12 near a bifurcation including a first, second and third branch vessel. The first stent 20 is positioned in main vessel 12 and the first branch vessel such that the first side hole aligns with the ostium of the second branch vessel and the second side hole aligns with the third branch vessel.

A second stent, including a first and a second side hole, is then positioned through the first stent in main vessel 12 and into the second branch vessel by way of the first side hole in the first stent. The first side hole of the second stent is aligned with the ostium of the first branch vessel and the second side hole is aligned with the ostium of the third branch vessel.

Next, a third stent, including a first and a second side hole, is positioned through the first and the second stent in main vessel 12 and into the third branch vessel by way of the aligned second side holes in the first and second stents. The first side hole of the second stent is aligned with the ostium of the first branch vessel and the second side hole is aligned with the ostium of the second branch vessel. Thus, all three branch vessels along with the main vessel are stented. In such a configuration, the three overlapped proximal portions each preferably have about one-third the mass as a corresponding distal portion.

In light of the foregoing description, it should be appreciated that any number of branch vessels could be stented according to the present invention. Further, it should be appreciated that many methods and sequences for positioning and deploying stents 20A and 20B may be provided according to the present invention. For example, U.S. patent application Ser. No. ______ (Attorney Docket No. 19601-000320), the entire disclosure of which is incorporated herein by reference, describes methods and apparatus for positioning and deploying stents near bifurcations. Further, the referenced application contains details related to aligning stent side holes with ostium of branch vessels. The methods and embodiments provided can be used in accordance with the present invention.

For example, the stent delivery system according to the present invention may employ a moveable or non-moveable side sheath or side member as further described in U.S. application Ser. No. ______ (Attorney Docket No. 19601-000320), the complete disclosure of which has been previously incorporated by reference. Additionally, for illustration, one embodiment of the referenced application provides an embodiment where a catheter system facilitates placement of the stent within the main vessel, with the side hole being in registry with an ostium of a branch vessel. This placement may be accomplished, for example, by advancing a main vessel guidewire in the main vessel until passing the branch vessel. The catheter is then advanced over the main vessel guidewire until the stent reaches or is proximal to the branch vessel. At this point, a branch vessel guidewire may be introduced through the branch vessel lumen of the catheter. The branch vessel guidewire is advanced out of the catheter and into the branch vessel to assist in aligning the side hole with the ostium of the branch vessel prior to deployment of the stent in the main vessel. To assist in guiding the branch vessel guidewire into the branch vessel, the catheter may taper at a point to a narrow distal end, which may also be curved slightly outwardly. One advantage of such a catheter system is that a single guidewire may be used to introduce the catheter. Once introduced, the catheter serves as a guide for the branch vessel guidewire.

Alignment of the side hole with the ostium can be accomplished in a variety of ways. For example, introduction of the branch vessel guidewire into the branch vessel may sufficiently align the side hole with the ostium. Other alignment techniques may depend on the configuration of the catheter. For example, in some cases the catheter may comprise a flexible sheath that is movably coupled to the catheter body, e.g., by passing through a lumen of a truncated connector that is coupled to the catheter body. Once the branch vessel guidewire is advanced into the branch vessel, the sheath may be advanced into the branch vessel to move the side hole into registry with the ostium.

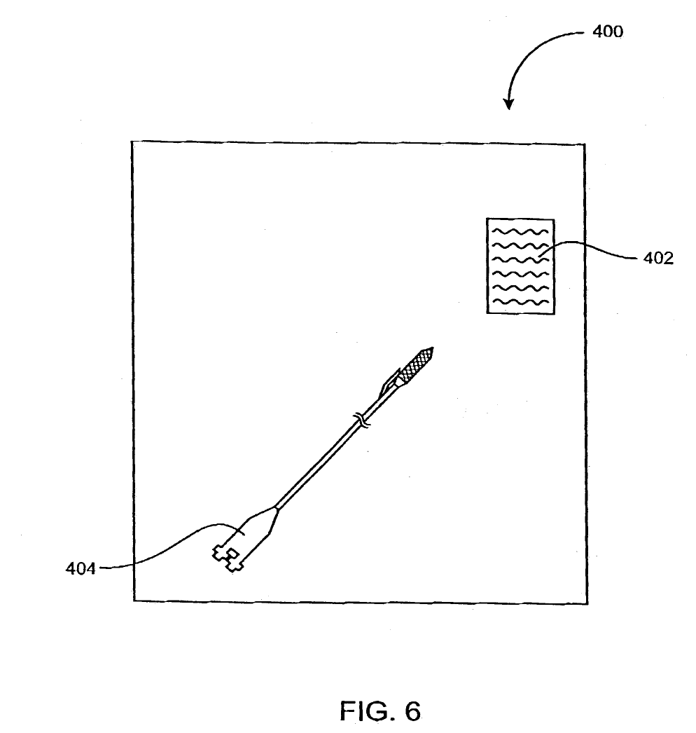

As shown in FIG. 6, a stent 20 may be conveniently included as part of a kit 400. Conveniently, kit 400 may include most any combination of apparatus and systems discussed herein, along with instructions for use 402 setting forth appropriate procedures for deploying stents using any of the techniques previously described. Instructions for use 402 may be written or in machine readable form. For example, kit 400 may include two stents, 20A and 20B, each crimped over a balloon 25 and coupled to the stent delivery system. The stent delivery system may include catheter 404, a side sheath or member, and/or a proximal hub, among other elements described or incorporated herein. Further, it will be appreciated that kit 400 may alternatively include any of the other elements described or incorporated herein, and instructions 402 may describe use of any of the other elements.

The invention has now been described in detail for purposes of clarity of understanding. However, it will be appreciated that certain changes and modifications may be practiced within the scope of the appended claims.

What is claimed is:

1 A stent for placement in a bifurcated body lumen, said stent comprising:
a tubular body defining a lumen therethrough and having a side hole, said body having a first portion with a first wall mass and a second portion with a second wall mass, wherein said first wall mass is less than said second wall mass.

2. The stent as in claim 1 wherein said first wall mass is about one-half said second wall mass.

3. The stent as in claim 1 wherein said first portion has a first cell density and said second portion has a second cell density, wherein said first cell density is less than said second cell density.

4. The stent as in claim 3 wherein said first cell density is about one half said second cell density.

5. The stent as in claim 1 wherein said first portion comprises a first plurality of struts and said second portion comprises a second plurality of struts, at least some of said first plurality of struts having a smaller cross-sectional area than said second plurality of struts.

6. The stent as in claim 1 wherein said first portion comprises a first plurality of struts and said second portion comprises a second plurality of struts, said second plurality of struts defining a greater cell density than said first plurality of struts.

7. The stent as in claim 1 wherein said first portion comprises said side hole.

8. The stent as in claim I wherein said first portion and said second portion have first and second wall thickness, respectively, said first wall thickness being less than said second wall thickness.

9. The stent as in claim 8 wherein said first wall thickness is about one-half said second wall thickness.

10. A stent system for placement in a bifurcated body lumen having a main vessel and first and second branch vessels, said system comprising:
a first tubular stent body having a first portion and a second portion, said first stent body having a side hole; and
a second tubular stent body having a first portion and a second portion, said second stent body having a side hole;
wherein said first stent body first portion is disposed within and generally coaxially aligned with said second stent body first portion, and said first stent body second portion extends through said side hole of said second stent body.

11. The stent system as in claim 10 wherein said combined first portions comprise about a same mass as one of said second portions.

12. The stent system as in claim 10 wherein said combined first portions comprise a cell density that is about equal to a cell density of one of said second portions.

13. The stent system as in claim 10 wherein said combined first portions have a wall thickness that is about equal to a wall thickness of one of said second portions.

14. The stent system as in claim 10 wherein said combined first portions are adapted to be positioned in said main vessel, said first stent body second portion is adapted to be positioned in said first branch vessel and said second stent body second portion is adapted to be positioned in said second branch vessel.

15. A method for deploying a stent in a bifurcated body lumen having a main vessel and first and second branch vessels, said method comprising:
providing first and second tubular stent bodies each having a first portion, a second portion, and a side hole;
positioning said first stent body within said bifurcated lumen so that said first portion is positioned within said main vessel and said second portion is positioned within said first branch vessel, said positioning further comprising alignment of said first stent body side hole with an ostium of said second branch vessel;
expanding said first stent body.
positioning said second stent body so that said first portion is generally aligned with and within said first stent body first portion in said main vessel, and said second portion extends through said first stent body side hole and into said second branch vessel, said positioning further comprising alignment of said second stent body side hole with an ostium of said first branch vessel; and
expanding said second stent body.

16. The method of claim 15 wherein said expanding said first stent body is performed prior to said positioning said second stent body.

17. The method of claim 15 wherein said combined first portions have about a same mass as one of said second portions.

18. The method of claim 15 wherein said combined first portions have about a same cell density as one of said second portions.

19. The method of claim 15 wherein said first and second stent bodies comprise a metal.

20. A kit comprising:
a stent as in claim 1; and
instructions for use setting forth a method for positioning said stent in a bifurcated body lumen having a main vessel and first and second branch vessels.
